SEQUENCE LISTING (45 sequences)

SEQ ID NO: 1 - protein, 7 aa, Artificial sequence
  Synthetic peptide
  MISC_FEATURE (5)..(5): X is 2-Aminoisobutyric acid (Aib)
LPPLXYP

SEQ ID NO: 2 - protein, 7 aa, Artificial sequence
  Synthetic peptide
  MISC_FEATURE (7)..(7): X is 2-Aminoisobutyric acid (Aib)
LPPLPYX

SEQ ID NO: 3 - protein, 7 aa, Artificial sequence
  Synthetic peptide
  MISC_FEATURE (1)..(1): X is D-leucine
  MISC_FEATURE (5)..(5): X is 2-Aminoisobutyric acid (Aib)
  MISC_FEATURE (7)..(7): X is 2-Aminoisobutyric acid (Aib)
  MISC_FEATURE (7)..(7): C terminal amidated
XPPLXYX

SEQ ID NO: 4 - protein, 5 aa, Artificial sequence
  Synthetic peptide
  MISC_FEATURE (1)..(1): X is D-proline
  MISC_FEATURE (4)..(4): X is 2-Aminoisobutyric acid (Aib)
  MISC_FEATURE (5)..(5): C terminal amidated
XPLXY

SEQ ID NO: 5 - protein, 6 aa, Artificial sequence
  Synthetic peptide
  MISC_FEATURE (1)..(1): X is D-leucine
  MISC_FEATURE (5)..(5): X is 2-Aminoisobutyric acid (Aib)
  MISC_FEATURE (6)..(6): C terminal amidated
XPPHXY

SEQ ID NO: 6 - protein, 7 aa, Artificial sequence
  Synthetic peptide
  MISC_FEATURE (4)..(4): X is D-leucine
LPPXPYP

SEQ ID NO: 7 - protein, 7 aa, Artificial sequence
  Synthetic peptide
  MISC_FEATURE (2)..(2): dimethylproline
LPPLPYP

SEQ ID NO: 8 - protein, 7 aa, Artificial sequence
  Synthetic peptide
LPPVPYP

SEQ ID NO: 9 - protein, 7 aa, Artificial sequence
  Synthetic peptide
LPPSPYP

SEQ ID NO: 10 - protein, 7 aa, Artificial sequence
  Synthetic peptide
LPPHPYP

SEQ ID NO: 11 - protein, 7 aa, Artificial sequence
  Synthetic peptide
  MISC_FEATURE (1)..(1): X is D-leucine
XPPHPYP

SEQ ID NO: 12 - protein, 7 aa, Artificial sequence
  Synthetic peptide
  MISC_FEATURE (1)..(1): X is D-leucine
  MISC_FEATURE (3)..(3): X is 2-Aminoisobutyric acid (Aib)
XPXLAYP

SEQ ID NO: 13 - protein, 7 aa, Artificial sequence
  Synthetic peptide
  MISC_FEATURE (3)..(3): dimethylproline
LPPLPYP

SEQ ID NO: 14 - protein, 7 aa, Artificial sequence
  Synthetic peptide
  MISC_FEATURE (3)..(3): X is 2-Aminoisobutyric acid (Aib)
LPXLPYP

SEQ ID NO: 15 - protein, 7 aa, Artificial sequence
  Synthetic peptide
  MISC_FEATURE (2)..(2): X is 2-Aminoisobutyric acid (Aib)
LXPLPYP

SEQ ID NO: 16 - protein, 7 aa, Artificial sequence
  Synthetic peptide
LPPAPYP

SEQ ID NO: 17 - protein, 7 aa, Artificial sequence
  Synthetic peptide
  MISC_FEATURE (7)..(7): X is D-proline
LPPLPYX

SEQ ID NO: 18 - protein, 7 aa, Artificial sequence
  Synthetic peptide
  MISC_FEATURE (1)..(1): Acetylated
LPPLPYP

SEQ ID NO: 19 - protein, 7 aa, Artificial sequence
  Synthetic peptide
LPPNPYP

SEQ ID NO: 20 - protein, 5 aa, Artificial sequence
  Synthetic peptide
PPLAY

SEQ ID NO: 21 - protein, 7 aa, Artificial sequence
  Synthetic peptide
LPPLYYP

SEQ ID NO: 22 - protein, 7 aa, Artificial sequence
  Synthetic peptide
LPPLHYP

SEQ ID NO: 23 - protein, 7 aa, Artificial sequence
  Synthetic peptide
LPPLLYP

SEQ ID NO: 24 - protein, 7 aa, Artificial sequence
  Synthetic peptide
LPPLPFP

SEQ ID NO: 25 - protein, 7 aa, Artificial sequence
  Synthetic peptide
  MISC_FEATURE (1)..(1): X is D-arginine
XPPLPYP

SEQ ID NO: 26 - protein, 7 aa, Artificial sequence
  Synthetic peptide
LPPTPYP

SEQ ID NO: 27 - protein, 7 aa, Artificial sequence
  Synthetic peptide
LPPLPWP

SEQ ID NO: 28 - protein, 7 aa, Artificial sequence
  Synthetic peptide
  MISC_FEATURE (6)..(6): X is D-valine
LPPLPXP

SEQ ID NO: 29 - protein, 7 aa, Artificial sequence
  Synthetic peptide
  MISC_FEATURE (6)..(6): X is D-aspartic acid
LPPLPXP

SEQ ID NO: 30 - protein, 5 aa, Artificial sequence
  Synthetic peptide
PPHPY

SEQ ID NO: 31 - protein, 7 aa, Artificial sequence
  Synthetic peptide
LPPHPYP

SEQ ID NO: 32 - protein, 7 aa, Artificial sequence
  Synthetic peptide
  MISC_FEATURE (1)..(1): X is D-leucine
XPPHAYP

SEQ ID NO: 33 - protein, 7 aa, Artificial sequence
  Synthetic peptide
  MISC_FEATURE (1)..(1): X is D-leucine
  MISC_FEATURE (2)..(2): X is 2-Aminoisobutyric acid (Aib)
XXPLAYP

SEQ ID NO: 34 - protein, 7 aa, Artificial sequence
  Synthetic peptide
  MISC_FEATURE (3)..(3): X is D-proline
LPXLPYP

SEQ ID NO: 35 - protein, 7 aa, Artificial sequence
  Synthetic peptide
  MISC_FEATURE (5)..(5): X is 2-Aminoisobutyric acid (Aib)
LPPHXYP

SEQ ID NO: 36 - protein, 7 aa, Artificial sequence
  Synthetic peptide
  MISC_FEATURE (1)..(1): X is D-leucine
  MISC_FEATURE (5)..(5): X is 2-Aminoisobutyric acid (Aib)
XPPHXYP

SEQ ID NO: 37 - protein, 5 aa, Artificial sequence
  Synthetic peptide
  MISC_FEATURE (1)..(1): Acetylated
  MISC_FEATURE (4)..(4): X is 2-Aminoisobutyric acid (Aib)
  MISC_FEATURE (5)..(5): C terminal amidated
PPLXY

SEQ ID NO: 38 - protein, 7 aa, Artificial sequence
  Synthetic peptide
  MISC_FEATURE (1)..(1): X is D-leucine
  MISC_FEATURE (5)..(5): X is 2-Aminoisobutyric acid (Aib)
XPPLXYP

SEQ ID NO: 39 - protein, 6 aa, Artificial sequence
  Synthetic peptide
  MISC_FEATURE (1)..(1): X is D-leucine
  MISC_FEATURE (5)..(5): X is 2-Aminoisobutyric acid (Aib)
XPPLXY

SEQ ID NO: 40 - protein, 6 aa, Artificial sequence
  Synthetic peptide
  MISC_FEATURE (1)..(1): X is D-leucine
XPPLAY

SEQ ID NO: 41 - protein, 7 aa, Artificial sequence
  Synthetic peptide
  MISC_FEATURE (1)..(1): X is D-leucine
  MISC_FEATURE (7)..(7): X is 2-Aminoisobutyric acid (Aib)
XPPLAYX

SEQ ID NO: 42 - protein, 7 aa, Artificial sequence
  Synthetic peptide
LPPLPYP

SEQ ID NO: 43 - protein, 5 aa, Artificial sequence
  Synthetic peptide
PPLPY

SEQ ID NO: 44 - protein, 7 aa, Artificial sequence
  Synthetic peptide
LPPLAYP

SEQ ID NO: 45 - protein, 7 aa, Artificial Sequence
  Synthetic peptide
  MISC_FEATURE (1)..(1): X is either  leucine, d-leucine, d-valine, d-arginine or absent
  MISC_FEATURE (2)..(2): X is either dimethylproline (dMP), proline, a-aminoisobutyric acid (Aib) or d-proline;
  MISC_FEATURE (3)..(3): X is either dimethylproline (dMP),  proline, Aib or d-proline
  MISC_FEATURE (4)..(4): X is either histidine, serine, valine, leucine, d-leucine or threonine
  MISC_FEATURE (5)..(5): X is either proline or alanine
  MISC_FEATURE (6)..(6): X is either tyrosine, d-valine, d-aspartic acid, tryptophan or phenylalanine
  MISC_FEATURE (7)..(7): X is either proline, dMP, d-proline or absent
XXXXXXX

(19) United States
(12) Patent Application Publication
(10) Pub. No.: US 2023/0049549 A1
(43) Pub. Date: Feb. 16, 2023

(54) PEPTIDE COMPOUNDS AND METHODS OF TREATING DISEASES USING SAME

(71) Applicant: Immunity Pharma Ltd., Mevasseret Zion (IL)

(72) Inventors: Eran OVADIA, Mevasseret Zion (IL); Avi BEN-SHIMON, Ayanot (IL); Ilana COHEN, Nes Ziona (IL)

(73) Assignee: Immunity Pharma Ltd., Mevasseret Zion (IL)

(21) Appl. No.: 17/792,787

(22) PCT Filed: Jan. 14, 2021
(86) PCT No.: PCT/IL2021/050044

§ 371 Date: Jul. 14, 2022

(30) Foreign Application Priority Data
Jan. 15, 2020 (IL) 272074

(51) Int. Cl. A61K 38/08; A61P 37/00

(52) U.S. Cl. CPC A61K 38/08; A61P 37/00

(57) ABSTRACT

Isolated peptides capable of reducing the amount of dexamethasone-induced spleen and/or thymus weight loss in a mouse are disclosed. Uses thereof for treating inflammatory or degenerative diseases are also disclosed.

### RELATED APPLICATION

This application claims the benefit of priority of Israeli Application No. 272074 filed on 15 Jan. 2020, the contents of which are incorporated herein by reference in their entirety.

### SEQUENCE LISTING STATEMENT

The ASCII file, entitled 84727 SequenceListing.txt, created on 13 Jan. 2021, comprising 12,288 bytes, submitted concurrently with the filing of this application is incorporated herein by reference.

### FIELD AND BACKGROUND OF THE INVENTION

The present invention, in some embodiments thereof, relates to compositions and methods of using same for treating inflammatory and autoimmune diseases.

There is an unmet need for novel compositions that may serve to attenuate cellular and immune stress-response in normal tissue, in a manner that is specific, safe and effective, thereby reducing the severity of stress associated degenerative diseases and stress-induced inflammation.

The peptide LPPLPYP (SEQ ID NO: 42, also known as Stressin-1 and IPL344) is a short 7 amino acids peptide that protects cells of various types from pro-apoptotic pressures and activates the Akt signaling system. The structure of IPL344 resembles the binding sites of adaptor proteins. It has been proposed to have a mechanism of action which comprises mimicking such proteins and activating cell protective processes via Akt and possibly other pathways.

International Patent Application Publication Nos: WO 2006/021954 and WO2012/160563 disclose the use of LPPLPYP (SEQ ID NO: 42) peptide for treating diseases such as ALS.

### SUMMARY OF THE INVENTION

According to an aspect of the present invention there is provided an isolated peptide being five or seven amino acids which consists of an amino acid sequence represented by the formula X₁-X₂-X₃-X₄-X₅-X₆-X₇ (SEQ ID NO: 45), wherein

(i) X₁, is selected from the group consisting of leucine, d-leucine, d-valine, d-arginine and absent;

(ii) X₂ is selected from the group consisting of dimethylproline (dMP), proline, α-aminoisobutyric acid (Aib) and d-proline;

(iii) X₃ is selected from the group consisting of dMP, proline Aib and d-proline;

(iv) X₄ is selected from the group consisting of histidine, serine, valine, leucine, d-leucine and threonine;

(v) X₅ is proline or alanine;

(vi) X₆ is selected from the group consisting of tyrosine, d-valine, d-aspartic acid, tryptophan and phenylalanine; and

(vii) X₇ is selected from the group consisting of proline, dMP, d-proline and absent the peptide is capable of reducing the amount of dexamethasone-induced spleen and/or thymus weight loss in a mouse, with the proviso that the peptide does not consist of the sequence as set forth in SEQ ID NOs 42, 43 or 44.

According to an aspect of the present invention there is provided a pharmaceutical composition comprising the peptide disclosed herein as an active agent and a physiologically acceptable carrier.

According to an aspect of the present invention there is provided an isolated peptide being no longer than ten amino acids which comprises an amino acid sequence selected from the group consisting of SEQ ID NOs: 1-33 and 34, wherein the peptide is capable of reducing the amount of dexamethasone-induced spleen and/or thymus weight loss in a mouse.

According to embodiments of the present invention, the peptide consists of the amino acid sequence selected from the group consisting of SEQ ID NOs: 6-33 and 34.

According to embodiments of the present invention, the peptide consists of the amino acid sequence selected from the group consisting of SEQ ID NOs: 6-12 and 13.

According to embodiments of the present invention, the peptide comprises an amino acid sequence selected from the group consisting of SEQ ID NOs: 1-4 and 5.

According to embodiments of the present invention, the peptide consists of the amino acid sequence selected from the group consisting of SEQ ID NOs: 1-33 and 34.

According to embodiments of the present invention, the peptide is a stapled peptide.

According to embodiments of the present invention, the peptide is a cyclic peptide.

According to embodiments of the present invention, the order of the sequence is reversed and all of the amino acids are of the D-type.

According to embodiments of the present invention, the peptide is attached to a cell penetrating moiety.

According to embodiments of the present invention, the cell penetrating moiety is attached to an N-terminus of the peptide.

According to embodiments of the present invention, the peptide is for use in treating a disease associated with apoptosis.

According to embodiments of the present invention, the disease associated with apoptosis is an inflammatory or degenerative disease.

According to embodiments of the present invention, the inflammatory disease is an autoimmune disease.

According to embodiments of the present invention, the degenerative disease is a neurodegenerative disease.

According to embodiments of the present invention, the disease associated with apoptosis is selected from the group consisting of age-related macular degeneration (AMD), retinitis pigmentosa, stroke and myocardial infarction.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### DESCRIPTION OF SPECIFIC EMBODIMENTS OF THE INVENTION

The present invention, in some embodiments thereof, relates to compositions and methods of using same for treating inflammatory and autoimmune diseases.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The multi-proline peptide LPPLPYP (SEQ ID NO: 42), also known as IPL344 and Stressin-1) is a short 7 amino acids peptide that protects cells of various types from pro-apoptotic pressures and activates the Akt signaling system. It is a candidate for treating degenerative, inflammatory and autoimmune diseases.

Whilst researching the contribution of the individual amino acids of the peptide, the present inventors surprisingly found that particular amino acid replacements of the core sequence showed a significant improvement in reduction of the amount of dexamethasone-induced spleen and/or thymus weight loss in mice, whereas other replacements and/or deletions severely diminished the reduction. In addition, these peptides were shown to minimize the decrease in dexamethasone induced-spleen and thymus cell number.

The present inventors found that the majority of these peptides conformed to the formula as set forth in SEQ ID NO: 45 and propose the use of such peptides for treating degenerative, inflammatory and autoimmune diseases.

Whilst further reducing the present invention to practice the present inventors noted that replacements of proline with the synthetic amino acid Aib at particular locations also led to peptides having enhanced improvement in reduction of the amount of dexamethasone-induced spleen and/or thymus weight loss in mice and minimizing the decrease in dexamethasone induced-spleen and thymus cell number.

The term “peptide” as used herein refers to a polymer of natural or synthetic amino acids, encompassing native peptides (either degradation products, synthetically synthesized polypeptides or recombinant polypeptides) and peptidomimetics (typically, synthetically synthesized peptides), as well as peptoids and semipeptoids which are polypeptide analogs, which may have, for example, modifications rendering the peptides even more stable while in a body or more capable of penetrating into cells.

The present invention also covers derivatives (with modification and/or addition of a chemical function to the amino acid side chain, without a chemical change in the peptidic backbone) and analogues (with modification and/or addition of a chemical function within the peptidic backbone, for example, an N-terminus or C-terminus modification or a peptide bond modification.

Such modifications include, but are not limited to N terminus modification, C terminus modification, polypeptide bond modification, including, but not limited to, CH2-NH, CH2-S, CH2-S═O, O═C—NH, CH2-O, CH2-CH2, S═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Polypeptide bonds (—CO—NH—) within the polypeptide may be substituted, for example, by N-methylated bonds (—N(CH3)-CO—), ester bonds (—C(R)H—C—O—O—C(R)—N—), ketomethylen bonds (—CO—CH2-), α-aza bonds (—NH—N(R)—CO—), wherein R is any alkyl, e.g., methyl, carba bonds (—CH2-NH—), hydroxyethylene bonds (—CH(OH)—CH2-), thioamide bonds (—CS—NH—), olefinic double bonds (—CH═CH—), retro amide bonds (—NH—CO—), polypeptide derivatives (—N(R)—CH2-CO—), wherein R is the “normal” side chain, naturally presented on the carbon atom.

These modifications can occur at any of the bonds along the polypeptide chain and even at several (2-3) at the same time.

Non-natural amino acids are summarized in Table 2, herein below.

As used herein in the specification and in the claims section below the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids (stereoisomers).

Tables 1 and 2 below list naturally occurring amino acids (Table 1) and non-conventional or modified amino acids (Table 2) which can be used with the present invention.

TABLE 1

| Amino | Three-Letter | One-letter |
|---|---|---|
| Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid | Xaa | X |
| as above | | |

TABLE 2

| Non-conventional amino acid | Code | Non-conventional amino acid | Code |
|---|---|---|---|
| ornithine | Orn | hydroxyproline | Hyp |
| α-aminobutyric acid | Abu | aminonorbomyl-carboxylate | Norb |
| D-alanine | Dala | aminocyclopropane-carboxylate | Cpro |
| D-arginine | Darg | N-(3-guanidinopropyl)glycine | Narg |
| D-asparagine | Dasn | N-(carbamylmethyl)glycine | Nasn |
| D-aspartic acid | Dasp | N-(carboxymethyl)glycine | Nasp |
| D-cysteine | Dcys | N-(thiomethyl)glycine | Ncys |
| D-glutamine | Dgln | N-(2-carbamylethyl)glycine | Ngln |
| D-glutamic acid | Dglu | N-(2-carboxyethyl)glycine | Nglu |
| D-histidine | Dhis | N-(imidazolylethyl)glycine | Nhis |
| D-isoleucine | Dile | N-(1-methylpropyl)glycine | Nile |
| D-leucine | Dleu | N-(2-methylpropyl)glycine | Nleu |
| D-lysine | Dlys | N-(4-aminobutyl)glycine | Nlys |
| D-methionine | Dmet | N-(2-methylthioethyl)glycine | Nmet |
| D-ornithine | Dorn | N-(3-aminopropyl)glycine | Norn |
| D-phenylalanine | Dphe | N-benzylglycine | Nphe |
| D-proline | Dpro | N-(hydroxymethyl)glycine | Nser |
| D-serine | Dser | N-(1-hydroxyethyl)glycine | Nthr |
| D-threonine | Dthr | N-(3-indolylethyl)glycine | Nhtrp |
| D-tryptophan | Dtrp | N-(p-hydroxyphenyl)glycine | Ntyr |
| D-tyrosine | Dtyr | N-(1-methylethyl)glycine | Nval |
| D-valine | Dval | N-methylglycine | Nmgly |
| D-N-methylalanine | Dnmala | L-N-methylalanine | Nmala |
| D-N-methylarginine | Dnmarg | L-N-methylarginine | Nmarg |
| D-N-methylasparagine | Dnmasn | L-N-methylasparagine | Nmasn |
| D-N-methylasparatate | Dnmasp | L-N-methylaspartic acid | Nmasp |
| D-N-methylcysteine | Dnmcys | L-N-methylcysteine | Nmcys |
| D-N-methylglutamine | Dnmgln | L-N-methylglutamine | Nmgln |
| D-N-methylglutamate | Dnmglu | L-N-methylglutamic acid | Nmglu |
| D-N-methylhistidine | Dnmhis | L-N-methylhistidine | Nmhis |
| D-N-methylisoleucine | Dnmile | L-N-methylisolleucine | Nmile |
| D-N-methylleucine | Dnmleu | L-N-methylleucine | Nmleu |
| D-N-methyllysine | Dnmlys | L-N-methyllysine | Nmlys |
| D-N-methylmethionine | Dnmmet | L-N-methylmethionine | Nmmet |
| D-N-methylomithine | Dnmorn | L-N-methylornithine | Nmorn |
| D-N-methylphenylalanine | Dnmphe | L-N-methylphenylalanine | Nmphe |
| D-N-methylproline | Dnmpro | L-N-methylproline | Nmpro |
| D-N-methylserine | Dnmser | L-N-methylserine | Nmser |
| D-N-methylthreonine | Dnmthr | L-N-methylthreonine | Nmthr |
| D-N-methyltryptophan | Dnmtrp | L-N-methyltryptophan | Nmtrp |
| D-N-methyltyrosine | Dnmtyr | L-N-methyltyrosine | Nmtyr |
| D-N-methylvaline | Dnmval | L-N-methylvaline | Nmval |
| L-norleucine | Nle | L-N-methylnorleucine | Nmnle |
| L-norvaline | Nva | L-N-methylnorvaline | Nmnva |
| L-ethylglycine | Etg | L-N-methyl-ethylglycine | Nmetg |
| L-t-butylglycine | Tbug | L-N-methyl-t-butylglycine | Nmtbug |
| L-homophenylalanine | Hphe | L-N-methyl-homophenylalanine | Nmhphe |
| α-naphthylalanine | Anap | N-methyl-α-naphthylalanine | Nmanap |
| penicillamine | Pen | N-methylpenicillamine | Nmpen |
| γ-aminobutyric acid | Gabu | N-methyl-γ-aminobutyrate | Nmgabu |
| cyclohexylalanine | Chexa | N-methyl-cyclohexylalanine | Nmchexa |
| cyclopentylalanine | Cpen | N-methyl-cyelopentylalanine | Nmcpen |
| α-amino-α-methylbutyrate | Aabu | N-methyl-α-amino-α-methylbutyrate | Nmaabu |
| α-aminoisobutyric acid | Aib | N-methyl-α-aminoisobutyrate | Nmaib |
| D-α-methylarginine | Dmarg | L-α-methylarginine | Marg |
| D-α-methylasparagine | Dmasn | L-α-methylasparagine | Masn |
| D-α-methylaspartate | Dmasp | L-α-methylaspartate | Masp |
| D-α-methylcysteine | Dmcys | L-α-methylcysteine | Mcys |
| D-α-methylglutamine | Dmgln | L-α-methylglutamine | Mgln |
| D-α-methyl glutamic acid | Dmglu | L-α-methylglutamate | Mglu |
| D-α-methylhistidine | Dmhis | L-α-methylhistidine | Mhis |
| D-α-methylisoleucine | DmIle | L-α-methylisoleucine | Mile |
| D-α-methylleucine | Dmleu | L-α-methylleucine | Mleu |
| D-α-methyllysine | Dmlys | L-α-methyllysine | Mlys |
| D-α-methylmethionine | Dmmet | L-α-methylmethionine | Mmet |
| D-α-methylomithine | Dmorn | L-α-methylornithine | Morn |
| D-α-methylphenylalanine | Dmphe | L-α-methylphenylalanine | Mphe |
| D-α-methylproline | Dmpro | L-α-methylproline | Mpro |
| D-α-methylserine | Dmser | L-α-methylserine | Mser |
| D-α-methylthreonine | Dmthr | L-α-methylthreonine | Mthr |
| D-α-methyltryptophan | Dmtrp | L-α-methyltryptophan | Mtrp |
| D-α-methyltyrosine | Dmtyr | L-α-methyltyrosine | Mtyr |
| D-α-methylvaline | Dmval | L-α-methylvaline | Mval |
| N-cyclobutylglycine | Ncbut | L-α-methylnorvaline | Mnva |
| N-cycloheptylglycine | Nchep | L-α-methylethylglycine | Metg |
| N-cyclohexylglycine | Nchex | L-α-methyl-t-butylglycine | Mtbug |
| N-cyclodecylglycine | Ncdec | L-α-methyl-homophenylalanine | Mhphe |
| N-cyclododecylglycine | Ncdod | α-methyl-α-naphthylalanine | Manap |
| N-cyclooctylglycine | Ncoct | α-methylpenicillamine | Mpen |
| N-cyclopropylglycine | Ncpro | α-methyl-γ-aminobuty rate | Mgabu |
| N-cycloundecylglycine | Ncund | α-methyl-cyclohexylalanine | Mchexa |
| N-(2-aminoethyl)glycine | Naeg | α-methyl-cyclopentylalanine | Mcpen |
| N-(2,2-diphenylethyl)glycine | Nbhm | N-(N-(2,2-diphenylethyl)carbamylmethyl-glycine | Nnbhm |
| N-(3,3-diphenylpropyl)glycine | Nbhe | N-(N-(3,3-diphenylpropyl)carbamylmethyl-glycine | Nnbhe |
| 1-carboxy-1-(2,2-diphenylethylamino)cyclopropane | Nmbc | 1,2,3,4-tetrahydroisoquinoline-3-carboxylic acid | Tic |
| phosphoserine | pSer | phosphothreonine | pThr |
| phosphotyrosine | pTyr | O-methyl-tyrosine | |
| 2-aminoadipic acid | | hydroxylysine | |
| Dimethyl proline | dmp | | |

As mentioned, the N and C termini of the peptides of the present invention may be protected by functional groups. Suitable functional groups are described in Green and Wuts, “Protecting Groups in Organic Synthesis”, John Wiley and Sons, Chapters 5 and 7, 1991, the teachings of which are incorporated herein by reference. Preferred protecting groups are those that facilitate transport of the compound attached thereto into a cell, for example, by reducing the hydrophilicity and increasing the lipophilicity of the compounds.

These moieties can be cleaved in vivo, either by hydrolysis or enzymatically, inside the cell. Hydroxyl protecting groups include esters, carbonates and carbamate protecting groups. Amine protecting groups include alkoxy and aryloxy carbonyl groups, as described above for N-terminal protecting groups. Carboxylic acid protecting groups include aliphatic, benzylic and aryl esters, as described above for C-terminal protecting groups. In one embodiment, the carboxylic acid group in the side chain of one or more glutamic acid or aspartic acid residue in a peptide of the present invention is protected, preferably with a methyl, ethyl, benzyl or substituted benzyl ester.

Examples of N-terminal protecting groups include acyl groups (—CO—R1) and alkoxy carbonyl or aryloxy carbonyl groups (—CO—O—1), wherein R1 is an aliphatic, substituted aliphatic, benzyl, substituted benzyl, aromatic or a substituted aromatic group. Specific examples of acyl groups include acetyl, (ethyl)-CO—, n-propyl-CO—, iso-propyl-CO—, n-butyl-CO—, sec-butyl-CO—, t-butyl-CO—, hexyl, lauroyl, palmitoyl, myristoyl, stearyl, oleoyl phenyl-CO—, substituted phenyl-CO—, benzyl-CO— and (substituted benzyl)-CO—. Examples of alkoxy carbonyl and aryloxy carbonyl groups include CH3-O—CO—, (ethyl)-O—CO—, n-propyl-O—CO—, iso-propyl-O—CO—, n-butyl-O—CO—, sec-butyl-O—CO—, t-butyl-O—CO—, phenyl-O—CO—, substituted phenyl-O—CO— and benzyl-O—CO—, (substituted benzyl)-O—CO—. Adamantan, naphtalen, myristoleyl, tuluen, biphenyl, cinnamoyl, nitrobenzoy, toluoyl, furoyl, benzoyl, cyclohexane, norbornane, Z-caproic. In order to facilitate the N-acylation, one to four glycine residues can be present in the N-terminus of the molecule.

The carboxyl group at the C-terminus of the compound can be protected, for example, by an amide (i.e., the hydroxyl group at the C-terminus is replaced with —NH₂, —NHR₂ and —NR₂R₃) or ester (i.e. the hydroxyl group at the C-terminus is replaced with —OR₂). R₂ and R₃ are independently an aliphatic, substituted aliphatic, benzyl, substituted benzyl, aryl or a substituted aryl group. In addition, taken together with the nitrogen atom, R₂ and R₃ can form a C4 to C8 heterocyclic ring with from about 0-2 additional heteroatoms such as nitrogen, oxygen or sulfur.

Examples of suitable heterocyclic rings include piperidinyl, pyrrolidinyl, morpholino, thiomorpholino or piperazinyl. Examples of C-terminal protecting groups include —NH₂, —NHCH 3, —N(CH₃)₂, —NH(ethyl), —N(ethyl)₂, —N(methyl) (ethyl), —NH(benzyl), —N(C1-C4 alkyl)(benzyl), —NH(phenyl), —N(C1-C4 alkyl) (phenyl), —OCH 3, —O-(ethyl), —O-(n-propyl), —O-(n-butyl), —O-(iso-propyl), —O-(sec-butyl), —O-(t-butyl), —O-benzyl and —O-phenyl.

The peptides of the present invention may also comprise non-amino acid moieties, such as for example, hydrophobic moieties (various linear, branched, cyclic, polycyclic or hetrocyclic hydrocarbons and hydrocarbon derivatives) attached to the peptides; non-peptide penetrating agents;

various protecting groups, especially where the compound is linear, which are attached to the compound's terminals to decrease degradation. Chemical (non-amino acid) groups present in the compound may be included in order to improve various physiological properties such; decreased degradation or clearance; decreased repulsion by various cellular pumps, improve immunogenic activities, improve various modes of administration (such as attachment of various sequences which allow penetration through various barriers, through the gut, etc.); increased specificity, increased affinity, decreased toxicity and the like.

Attaching the amino acid sequence component of the peptides of the invention to other non-amino acid agents may be by covalent linking, by non-covalent complexion, for example, by complexion to a hydrophobic polymer, which can be degraded or cleaved producing a compound capable of sustained release; by entrapping the amino acid part of the peptide in liposomes or micelles to produce the final peptide of the invention. The association may be by the entrapment of the amino acid sequence within the other component (liposome, micelle) or the impregnation of the amino acid sequence within a polymer to produce the final peptide of the invention.

According to a particular embodiment, the peptide is attached to a cell penetrating moiety.

As used herein, the term “cell penetrating moiety” refers to a moiety (e.g. a lipid, such as palmitic acid) which enhances translocation of an attached peptide across a cell membrane. In a particular embodiment, the cell penetrating moiety is not a peptide moiety. The moiety may be attached to the N or to the C terminus.

The peptides of the invention may be linear or cyclic (cyclization may improve stability).

Cyclization may take place by any means known in the art. Where the compound is composed predominantly of amino acids, cyclization may be via N- to C-terminal, N-terminal to side chain and N-terminal to backbone, C-terminal to side chain, C-terminal to backbone, side chain to backbone and side chain to side chain, as well as backbone to backbone cyclization. Cyclization of the peptide may also take place through non-amino acid organic moieties comprised in the peptide.

The present inventors also conceive of stapled peptides.

The term “stapled peptide” as used herein refers to a peptide having a selected number of standard or non-standard amino acids, and further having at least two moieties capable of undergoing reaction to promote carbon-carbon bond formation, that has been contacted with a reagent to generate at least one cross-linker between the at least two moieties, which modulates, for example, peptide stability.

The term “stapling” as used herein introduces into a peptide at least two moieties capable of undergoing reaction to promote carbon-carbon bond formation that can be contacted with a reagent to generate at least one cross-linker between the at least two moieties. Stapling provides a constraint on a secondary structure, such as an .alpha.-helix structure. The length and geometry of the cross-linker can be optimized to improve the yield of the desired secondary structure content. The constraint provided can, for example, prevent the secondary structure from unfolding and/or can reinforce the shape of the secondary structure. A secondary structure that is prevented from unfolding is, for example, more stable.

The peptides of the present invention can be biochemically synthesized such as by using standard solid phase techniques. These methods include exclusive solid phase synthesis, partial solid phase synthesis methods, fragment condensation, classical solution synthesis. Solid phase polypeptide synthesis procedures are well known in the art and further described by John Morrow Stewart and Janis Dillaha Young, Solid Phase Polypeptide Syntheses (2nd Ed., Pierce Chemical Company, 1984).

Liquid phase techniques which are particularly suitable for small peptides are also contemplated by the present inventors.

Large scale peptide synthesis is described by Andersson Biopolymers 2000; 55(3):227-50. Synthetic peptides can be purified by preparative high performance liquid chromatography [Creighton T. (1983) Proteins, structures and molecular principles. WH Freeman and Co. N.Y.] and the composition of which can be confirmed via amino acid sequencing.

Recombinant techniques may also be used to generate the peptides of the present invention. To produce a peptide of the present invention using recombinant technology, a polynucleotide encoding the peptide of the present invention is ligated into a nucleic acid expression vector, which comprises the polynucleotide sequence under the transcriptional control of a cis-regulatory sequence (e.g., promoter sequence) suitable for directing constitutive, tissue specific or inducible transcription of the polypeptides of the present invention in the host cells.

In addition to being synthesizable in host cells, the peptides of the present invention can also be synthesized using in vitro expression systems. These methods are well known in the art and the components of the system are commercially available.

The peptides described herein are capable of reducing the amount of dexamethasone-induced spleen and/or thymus weight loss in a mouse—e.g. following injection (IP) with 100 μg of Dexamethasone. Furthermore, they are capable of minimizing the decrease in dexamethasone induced-spleen and thymus cell number.

In another embodiment, the peptides described herein are capable of interfering and blocking both TNF-α and IL-6 secretion by macrophage cells in response to innate activators such as lipopolysaccharide (LPS) and CpG oligonucleotides.

Additionally or alternatively, the peptides described herein are capable or reducing, preventing or inhibiting apoptosis in eukaryotic cells. Irrespective of the mechanism by which the peptides of the invention mediates stress responses, and without wishing to be bound by any theory or mechanism of action, it is postulated that the peptides may be capable of activating the Akt-CREB axis. The peptide may be tested by analyzing their capacity to activate Akt kinase and/or cAMP response element-binding protein (CREB) transcription factor, as further described in Herkel et al., Immunology, 2017, 151, pages 474-480, the contents of which are incorporated herein by reference.

Methods of measuring apoptosis: Apoptosis is an active, gene-directed self-destruction process of the cell and is associated with characteristic morphological and biochemical changes.

Nuclear and cytoplasmic condensation and fragmentation of the dying cell into membrane-bound apoptotic bodies are typical characteristics of apoptosis. Another feature of apoptotic cell death is the chromosomal DNA degradation into oligonucleosomal fragments after the activation of specific nucleases.

By “inhibiting apoptosis” or “inhibits apoptotic activity” is meant any decrease in the number of cells that undergo apoptosis relative to an untreated control (i.e. cells not exposed to the peptides of the invention). Preferably, the decrease is at least 25%, more preferably the decrease is at least 50%, more preferably the decrease is at least 65%, and most preferably the decrease is at least 80%.

Flow cytometry offers a wide variety of possibilities to measure apoptosis. Different methods have been established and implemented, some which stain on the cell surface and some which stain intracellularly.

One of the first approaches was, beside the observation that apoptotic cells shrink and have higher intracellular granularity, to stain with DNA specific fluorochromes (e.g. propidium iodide [PI], ethidium bromide [EtBr]). As soon as a lethal hit is being induced, the DNA starts to change its profile. Apoptotic DNA not only consists of fragmented DNA (visualized as shorter bands, so called DNA ladder, in an agarose gel) but is also partially digested into single nucleotides, so that fluorochromes, like PI or EtBr, have less DNA to stain (Nicoletti et al., 1991). This is typically observed by a shift to the left, called sub-G1 peak, on the specific fluorochrome detection channel in the FACScan™ (from Becton Dickinson, USA).

Another method is the terminal deoxynucleotidyl transferase (TdT)-mediated endlabeling of the DNA strand breaks (TUNEL). The TUNEL method detects DNA strand breaks in cells undergoing apoptosis. TdT is an enzyme which catalyzes the addition of deoxyribonucleotide triphosphate to the 3′-OH ends of double or single-stranded DNA. Unlike normal cells, apoptotic cell nuclei incorporate exogenous nucleotides (dUTP)-DIG in the presence of TdT. An anti-DIG antibody fragment with a conjugated fluorochrome enables the visualization of apoptotic cells. An increase of apoptotic cells causes a higher number of DNA fragments and consequently a brighter fluorescence. An advantage of this method is the very high specificity (Gavrieli et al., 1992). A disadvantage of this method is that it is expensive and can only be used for a small set of samples, because it is time intensive. Therefore, it is not applicable for large screening programs.

The loss of cell membrane polarity and the presentation of increased amounts of phosphatidyl serine (PS) on the outside of the cell membrane during the early phase of apoptosis has led to yet a new approach. Annexin V is a calcium-dependent phospholipid binding protein with high affinity for PS. The cell membrane integrity is maintained in the early and intermediate phases of apoptosis. Early and intermediate apoptotic cells show increased binding of Annexin-FITC and are mainly negative for PI-staining. Late apoptotic stages and necrotic cells become double positive, because of PS presentation on the surface and the PI staining of intracellular nucleic acids due to disintegration of the membrane. This method is also costly and labor intensive.

Other methods for measuring apoptosis in vivo and in vitro are disclosed in U.S. Pat. Nos. 6,726,895 and 6,723,567.

Thus, according to a first aspect of the present invention, there is provided an isolated peptide being five or seven amino acids which consists of an amino acid sequence represented by the formula X₁-X₂-X₃-X₄-X₅-X₆-X₇ (SEQ ID NO: 45), wherein
(i) X₁, is selected from the group consisting of leucine, d-leucine, d-valine, d-arginine and absent;

(ii) X₂ is selected from the group consisting of dMP, proline, Aib and d-proline; (iii) X₃ is selected from the group consisting of dMP, proline, Aib and d-proline; (iv) X₄ is selected from the group consisting of histidine, serine, valine, leucine, d-leucine and threonine;

(v) X₅ is proline or alanine;
(vi) X₆ is selected from the group consisting of tyrosine, d-valine, d-aspartic acid, tryptophan and phenylalanine; and
(vii) X₇ is selected from the group consisting of proline, dMP, d-proline and absent, the peptide is capable of reducing the amount of dexamethasone-induced spleen and/or thymus weight loss in a mouse, with the proviso that the peptide does not consist of the sequence as set forth in SEQ ID NOs 42, 43 or 44.

The peptide according to this aspect of the present invention may be five or 7 amino acids in length. Accordingly, when X₁ is absent, then X₇ is absent as well.

Examples of peptides that are contemplated by this aspect of the present invention are set forth in SEQ ID NOs: 6-34.

In one embodiment, the peptide consists of any one of the sequences set forth in SEQ ID NOs: 6-34.

In another embodiment, the peptide consists of one of the sequences set forth in SEQ ID NOs: 6-13.

It will be appreciated that for this aspect of the present invention, the amino acids are set forth in the formula according to SEQ ID NO: 45 and no conservative/non-conservative mutations other than those specified are considered. Furthermore, when a particular stereoisomer appears in the formula, it is clear that it cannot be replaced by the other stereoisomer.

According to another aspect of the present invention, there is provided an isolated peptide being no longer than ten amino acids which comprises an amino acid sequence selected from the group consisting of SEQ ID NOs: 1-33 and 34, wherein the peptide is capable of reducing the amount of dexamethasone-induced spleen and/or thymus weight loss in a mouse.

In one embodiment, the peptide of this aspect of the present is 10 amino acids in length.

In one embodiment, the peptide of this aspect of the present is 9 amino acids in length.

In one embodiment, the peptide of this aspect of the present is 8 amino acids in length.

In one embodiment, the peptide of this aspect of the present is 7 amino acids in length.

In one embodiment, the peptide of this aspect of the present is 6 amino acids in length.

In one embodiment, the peptide of this aspect of the present is 5 amino acids in length.

Examples of such peptides include those set forth in SEQ ID NOs: 1-34, and more specifically those set forth in SEQ ID NOs: 1-5.

For any of the peptides described herein, the present invention also contemplates retro-inverso peptides. Such peptides are resistant to proteases and consist of D-amino acids in reversed order, resulting in an altered peptide backbone but unchanged orientation of the side chains.

The peptides described herein may be used for treating a myriad of diseases including those associated with stress-associated responses. These include pathological conditions such as neurodegenerative diseases (e.g. stroke, Parkinson's, and Alzheimer's disease), myocardial infarction, exposure to radiation or chemotherapeutic agents, inflammation, injuries (e.g., burns and central nervous system injuries), cell aging, hyperthermia, seizures, hypoxias (e.g., ischemia and stroke), and in transplant tissues and organs prior to transplanting.

These conditions also include autoimmune diseases, characterized by a state of immunization of an individual against at least one of the body's normal constituents. These phenomena are observed in particular in pathologies including, but not limited to infections associated with SLE (Systemic Lupus Erythematosus disease), Gougerot-Sjogren syndrome (or Sjogren's disease) and rheumatoid polyarthritis, as well as pathologies such as sarcoidosis and osteopenia, spondyloarthritis, scleroderma, multiple sclerosis, amyotrophic lateral sclerosis (ALS), hyperthyroidism, Addison's disease, autoimmune hemolytic anemia, Crohn's disease, Goodpasture's syndrome, Graves' disease, Hashimoto's thyroiditis, idiopathic purpura hemorrhage, insulin-dependent diabetes, myasthenia, pemphigus vulgaris, pernicious anemia, poststreptococcal glomerulonephritis, psoriasis and spontaneous sterility, as well as immediate or delayed phenomena observed during graft rejections and graft-versus host disease. In another embodiment, the peptides of the invention are useful for the treatment of ischemia or myocardial infarction.

According to a particular embodiment, the disease is ALS.

Other diseases contemplated by the present invention include but not limited to, Alzheimer's disease, Parkinson's disease, secondary degeneration after trauma, stroke, CNS intoxication, glaucoma, macular degeneration, type 1 diabetes, systemic lupus erythematosis, autoimmune uveitis, graft versus host disease, graft rejection, arthritis, systemic inflammatory response syndrome (SIRS) inflammatory bowel disease (IBD), adult respiratory distress syndrome (ARDS), psoriasis, atherosclerosis, myocardial infarction, radiation disease, hyperthermia, hypoxia, fulminant toxic liver, kidney failure, infertility and many others.

The phenomenon of graft rejection is a state of immunization of an individual against foreign constituents (bodily fluids such as blood, cerebrospinal fluid, etc., cells, tissues, organs, antibodies, etc.) deliberately implanted into the patient.

As used herein, the terms “degenerative disorder” “degenerative disease” and “degenerative condition” are directed to any disorder, disease or condition characterized by inappropriate cell proliferation or inappropriate cell death or in some cases, both, or aberrant or disregulated apoptosis. These conditions also include conditions in which, although appropriate and regulated at the level of a single cell, excessive apoptosis is associated with organ dysfunction or failure.

In one embodiment, the peptides are useful to prevent cell death in non-malignant tissue or cells in a subject having a neoplastic disorder and undergoing chemotherapy and/or radiation therapy for the treatment of cancer.

The terms “inflammatory disease” and “inflammatory condition”, as used herein, mean any disease or condition in which an excessive or unregulated inflammatory response leads to excessive inflammatory symptoms, host tissue damage, or loss of tissue function.

In one embodiment, the inflammatory disease or condition is an autoimmune disease.

In another embodiment, the inflammatory disease or condition has an etiology associated with production of at least one pro-inflammatory cytokine selected from IL-6 and TNF-α.

In another embodiment, the disease or condition is selected from the group consisting of: Alzheimer's disease, Parkinson's disease, secondary degeneration after trauma, stroke, CNS intoxication, glaucoma, macular degeneration, myocardial infarction, radiation disease, hyperthermia, hypoxia, fulminant toxic liver, kidney failure and infertility.

In still another embodiment, the disease includes retinitis pigmentosa and macular degeneration.

In another embodiment, the disease includes stroke or myocardial infarction.

The peptides may be provided per se or as part of a pharmaceutical composition, where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the peptides accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

The preparation of pharmaceutical compositions, which contain peptides or polypeptides as active ingredients is well known in the art. Typically, such compositions are prepared as indictable, either as liquid solutions or suspensions, however, solid forms, which can be suspended or solubilized prior to injection, can also be prepared. The preparation can also be emulsified. The active therapeutic ingredient is mixed with inorganic and/or organic carriers, which are pharmaceutically acceptable and compatible with the active ingredient. Carriers are pharmaceutically acceptable excipients (vehicles) comprising more or less inert substances when added to a pharmaceutical composition to confer suitable consistency or form to the composition. Suitable carriers are, for example, water, saline, dextrose, glycerol, ethanol, or the like and combinations thereof. In addition, if desired, the composition can contain minor amounts of auxiliary substances such as wetting or emulsifying agents and pH buffering agents, which enhance the effectiveness of the active ingredient.

Toxicity and therapeutic efficacy of the peptides described herein can be determined by standard pharmaceutical procedures in cell cultures or experimental animals, e. g., by determining the IC₅₀ (the concentration which provides 50% inhibition) and the LD₅₀ (lethal dose causing death in 50% of the tested animals) for a subject compound. The data obtained from these cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl et al., 1975).

The amount of active agent used in an administration composition of the present invention is an amount effective to accomplish the purpose of the particular active agent for the target indication. The amount of active agent in the compositions typically is a pharmacologically, biologically, therapeutically, or chemically effective amount. However, the amount can be less than that amount when the composition is used in a dosage unit form because the dosage unit form may contain a plurality of compounds or active agents in a single composition or may contain a divided pharmacologically, biologically, therapeutically, or chemically effective amount. The total effective amount can then be administered in cumulative units containing, in total, an effective amount of the active agent.

A therapeutically effective amount of a peptide of the invention is an amount that when administered to a patient is capable of exerting an anti-apoptotic activity and/or an anti-inflammatory activity. Assays for detecting the anti-apoptotic activity of the peptide of the invention include, but are not limited to, staining DNA with specific fluorochromes such as propidium iodide and ethidium bromide, Annexin V assays, TUNEL assays and the like; certain non-limitative examples of such assays are presented in the Examples below. Assays for detecting anti-inflammatory activity of the peptides are also well known in the art.

Although an appropriate dosage of a peptide of the invention varies depending on the administration route, age, body weight sex or conditions of the patient, and should be determined by the physician in the end, the dose suitable for adult humans (e.g. when administered i.v.) can generally be between about 2-6 mg body weight, preferably between about 2-4 mg/kg.

The pharmaceutical compositions of the present invention comprises one or more compounds of the present invention, and one or more excipients or diluents. In one embodiment, one or more of the compounds, or solvates, or salts of these compounds.

The term “pharmaceutically acceptable salt” as used herein, refers to salts which are substantially non-toxic to living organisms. Typical pharmaceutically acceptable salts include those salts prepared by reaction of the compounds of the present invention with a pharmaceutically acceptable mineral or organic acid. Such salts are also known as acid addition salts.

The compositions comprising the compounds and active agents have utility in the delivery of active agents to selected biological systems and in an increased or improved bioavailability of the active agent compared to administration of the active agent without the delivery agent.

Delivery can be improved by delivering more active agent over a period of time, or in delivering active agent in a particular time period (such as to effect quicker or delayed delivery) or over a period of time (such as sustained delivery).

Pharmaceutical compositions for use in accordance with the present invention thus may be formulated in conventional manner using one or more physiologically acceptable carriers comprising excipients and auxiliaries, which facilitate processing of the active compounds into preparations which can be used pharmaceutically. Proper formulation is dependent upon the route of administration chosen.

The pharmaceutical compositions can be administered locally or systemically by any conventional and appropriate route including, but not limited to, oral, intraperitoneal, parenteral, intravenous, intramuscular, subcutaneous, transdermal, intrathecal, topical, rectal, buccal, inhalational or intranasal.

For injection, the compounds of the invention may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants for example DMSO, or polyethylene glycol are generally known in the art.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers.

In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for the chosen route of administration.

Alternatively, the compounds of the present invention can be incorporated into oral liquid preparations such as aqueous or oily suspensions, solutions, emulsions, syrups, or elixirs, for example. Moreover, formulations containing these compounds can be presented as a dry product for constitution with water or other suitable vehicle before use. Such liquid preparations can contain conventional additives, like suspending agents, such as sorbitol syrup, methyl cellulose, glucose/sugar syrup, gelatin, hydroxyethylcellulose, carboxymethyl cellulose, aluminum stearate gel, and hydrogenated edible fats; emulsifying agents, such as lecithin, sorbitan monooleate, or acacia; nonaqueous vehicles (which can include edible oils), such as almond oil, fractionated coconut oil, oily esters, propylene glycol, and ethyl alcohol; and preservatives, such as methyl or propyl p-hydroxybenzoate and sorbic acid.

For administration by inhalation, the peptides for use according to the present invention are conveniently delivered in the form of an aerosol spray presentation from a pressurized pack or a nebulizer with the use of a suitable propellant, e. g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the peptide and a suitable powder base such as lactose or starch.

The pharmaceutical compositions of the invention are also useful for topical and intralesional application. As used herein, the term “topical” means “pertaining to a particular surface area”, e.g. skin and mucosa, and the topical agent applied to a certain area of the surface will affect only the area to which it is applied. The formulations of the peptides/peptide analogs may be administered topically as a gel, ointment, cream, emulsion, sustained release formulation including a transdermal patch, and may comprise liposomes and any other pharmaceutically acceptable carrier suitable for administration of the drug topically. The pharmaceutical compositions herein described may also comprise suitable solid of gel phase carriers or excipients. Examples of such carriers or excipients include, but are not limited to, calcium carbonate, calcium phosphate, various sugars, starches, cellulose derivatives, gelatin and polymers such as polyethylene glycols.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

Methods:

Dexamethasone is a corticosteroid medication that induces apoptosis of immune cells and lymphomyeloid tissues. BALB/c mice were used to examine the ability of candidate peptides to rescue lymphocyte cells from apoptosis. Mice were injected IP with 100 μg of Dexamethasone. Dexamethasone-treated mice received immediately following and 24-hours after dexamethasone treatment IV injection of candidate peptides (200 μg peptide/mouse). Mice were sacrificed 48 hours after the first treatment. The spleen and the thymus were weighted and full cell count of both organs was performed.

Peptides used in the screen were SEQ ID NOs: 1-13. The peptide of SEQ ID NO: 42 was used as a positive control. The peptides set forth in SEQ ID NOs: 35-41 were used as negative controls.

Results:

The results are summarized in Table 3 and Table 4

TABLE 3

| | SEQ ID NO: | | | | | | | | | | | | | | | |
|---|---|---|---|---|---|---|---|---|---|---|---|---|---|---|---|---|
| | Normal | DEXA | 42 | 13 | 11 | 12 | 3 | 4 | 8 | 9 | 10 | 1 | 2 | 6 | 7 | 5 |
| Spleen | 100% | 62% | 79% | 82% | 81% | 89% | 85% | 77% | 76% | 76% | 84% | 89% | 89% | 80% | 84% | 81% |
| Weight | | | | | | | | | | | | | | | | |
| Spleen Cell | 100% | 59% | 78% | 84% | 91% | 85% | 79% | 82% | 82% | 82% | 85% | 82% | 85% | 80% | 82% | 91% |
| number | | | | | | | | | | | | | | | | |
| Thymus | 100% | 44% | 47% | 49% | 52% | 63% | 60% | 55% | 53% | 55% | 56% | 59% | 61% | 48 % | 72% | 52% |
| Weight | | | | | | | | | | | | | | | | |
| Thymus Cell | 100% | 23% | 34% | 44% | 49% | 51% | 33% | 31% | 30% | 31% | 33% | 28% | 25% | 23% | 29% | 49% |
| number | | | | | | | | | | | | | | | | |
| Average | 100% | 47% | 59% | 65% | 68% | 72% | 64% | 61% | 60% | 61% | 65% | 65% | 65% | 58% | 67% | 68% |

TABLE 4

| | SEQ ID NO: | | | | | | |
|---|---|---|---|---|---|---|---|
| | SEQ | SEQ | SEQ | SEQ | SEQ | SEQ | SEQ |
| | ID | ID | ID | ID | ID | ID | ID |
| | NO: | NO: | NO: | NO: | NO: | NO: | NO: |
| | 37 | 39 | 35 | 36 | 40 | 41 | 38 |
| Spleen | 69% | 69% | 62% | 64% | 72% | 69% | 67% |
| Weight | | | | | | | |
| Spleen | 77% | 73% | 65% | 69% | 72% | 61% | 66% |
| Cell | | | | | | | |
| number | | | | | | | |
| Thymus | 48% | 50% | 42% | 43% | 41% | 49% | 50% |
| Weight | | | | | | | |
| Thymus | 24% | 27% | 39% | 40% | 34% | 25% | 24% |
| Cell | | | | | | | |
| number | | | | | | | |
| Average | 55% | 55% | 52% | 54% | 55% | 51% | 52% |

Dexamethasone induced spleen and thymus weight loss and reduction in spleen cell count of about 50%. Thymus cell count was reduced by almost 60% compared to normal mice.

All the peptides in Table 3 showed a significant improvement over SEQ ID NO: 42. All the peptides in Table 4 had a reduced effect as compared to SEQ ID NO: 42.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In addition, the priority document of this application is hereby incorporated herein by reference in its entirety.

1. An isolated peptide being five or seven amino acids which consists of an amino acid sequence represented by the formula X₁-X₂-X₃-X₄-X₅-X₆-X₇ (SEQ ID NO: 45), wherein
(i) X₁, is selected from the group consisting of leucine, d-leucine, d-valine, d-arginine and absent;
(ii) X₂ is selected from the group consisting of dimethylproline (dMP), proline, α-aminoisobutyric acid (Aib) and d-proline;
(iii) X₃ is selected from the group consisting of dMP, proline Aib and d-proline;
(iv) X₄ is selected from the group consisting of histidine, serine, valine, leucine, d-leucine and threonine;
(v) X₅ is proline or alanine;
(vi) X₆ is selected from the group consisting of tyrosine, d-valine, d-aspartic acid, tryptophan and phenylalanine; and
(vii) X₇ is selected from the group consisting of proline, dMP, d-proline and absent the peptide is capable of reducing the amount of dexamethasone-induced spleen and/or thymus weight loss in a mouse, with the proviso that the peptide does not consist of the sequence as set forth in SEQ ID NOs 42, 43 or 44.

2. The isolated peptide of claim 1, consisting of the amino acid sequence selected from the group consisting of SEQ ID NOs: 6-33 and 34.

3. The isolated peptide of claim 1, consisting of the amino acid sequence selected from the group consisting of SEQ ID NOs: 6-12 and 13.

4. An isolated peptide being no longer than ten amino acids which comprises an amino acid sequence selected from the group consisting of SEQ ID NOs: 1-33 and 34, wherein the peptide is capable of reducing the amount of dexamethasone-induced spleen and/or thymus weight loss in a mouse.

5. The isolated peptide of claim 4, comprising an amino acid sequence selected from the group consisting of SEQ ID NOs: 1-4 and 5.

6. The isolated peptide of claim 4, consisting of the amino acid sequence selected from the group consisting of SEQ ID NOs: 1-33 and 34.

7. The isolated peptide of claim 1, wherein the peptide is a stapled peptide.

8. The isolated peptide of claim 1, wherein the peptide is a cyclic peptide.

9. The isolated peptide of claim 4, wherein the order of the sequence is reversed and all of the amino acids are of the D-type.

10. The isolated peptide of claim 1, wherein the peptide is attached to a cell penetrating moiety.

11. The isolated peptide of claim 10, wherein said cell penetrating moiety is attached to an N-terminus of the peptide.

12. A method of treating a disease associated with apoptosis in a subject in need thereof comprising administering a therapeutically effective amount of the isolated peptide of claim 1 to the subject, thereby treating the disease.

13. The method of claim 12, wherein said disease associated with apoptosis is an inflammatory or degenerative disease.

14. The method of claim 13, wherein the inflammatory disease is an autoimmune disease.

15. The method of claim 13, wherein said degenerative disease is a neurodegenerative disease.

16. The method of claim 12, wherein said disease associated with apoptosis is selected from the group consisting of age-related macular degeneration (AMD), retinitis pigmentosa, stroke and myocardial infarction.

17. A pharmaceutical composition comprising the peptide of claim 1 as an active agent and a physiologically acceptable carrier.
